SEQUENCE LISTING (2 sequences)

SEQ ID NO: 1 - protein, 14 aa, Homo sapiens
ALKRQGRTLYGFGG

SEQ ID NO: 2 - protein, 5 aa, Homo sapiens
YGFGG

(19) United States
(12) Patent Application Publication
(10) Pub. No.: US 2024/0285723 A1
(43) Pub. Date: Aug. 29, 2024

(54) COMBINATION COMPOSITIONS FOR THE TREATMENT OF PATHOGEN INFECTIONS AND ASSOCIATED COMPLICATIONS

(71) Applicant: RAMOT AT TEL-AVIV UNIVERSITY LTD., Tel Aviv (IL)

(72) Inventors: Yankel GABET, Tel Aviv (IL); Josef MOGRABI, Tel Aviv (IL)

(21) Appl. No.: 18/573,517

(22) PCT Filed: Jun. 22, 2022
(86) PCT No.: PCT/IL2022/050676

§ 371 Date: Dec. 22, 2023

Related U.S. Application Data
(60) Provisional application No. 63/214,273, filed on Jun. 24, 2021.

(51) Int. Cl. A61K 38/08; A61K 31/015; A61K 31/497; A61K 31/566; A61K 31/573; A61P 31/00; A61P 37/06

(52) U.S. Cl. CPC A61K 38/08; A61K 31/015; A61K 31/497; A61K 31/566; A61K 31/573; A61P 31/00; A61P 37/06

(57) ABSTRACT

Pharmaceutical compositions are provided, comprising at least one selective CB2 receptor agonist and at least one steroid. The provided compositions are effective in treating inflammation and/or pathogen related inflammation and associated complications thereof. Also provided are methods for treating a subject infected with a pathogen as well as inflammation, using said compositions.

### FIELD OF THE INVENTION

The present invention relates to compositions effective in treating pathogen related inflammation and associated complications thereof, the compositions comprising combinations of CB2 receptor agonists and steroids.

### BACKGROUND OF THE INVENTION

Coronaviruses (CoVs) are a large family of viruses belonging to the Coronavirinae subfamily. There are three coronaviruses that are known to cause serious life-threatening infections in people, namely Severe Acute Respiratory Syndrome Coronavirus (SARS-CoV), Middle East Respiratory Syndrome Coronavirus (MERS-COV), and Severe Acute Respiratory Syndrome Coronavirus 2 (SARS-COV-2), the latter was identified as the cause of a pandemic of the viral respiratory disease named COVID-19.

The COVID-19 pandemic has caught the scientific and medical communities unprepared to address the related clinical challenges of cytokine storm syndrome (CSS) and septic shock. Respiratory failure from acute respiratory distress syndrome (ARDS) is the leading cause of mortality in COVID-19 patients. ARDS will usually appear in COVID-19 patients one or two days following sepsis accompanied by classical serum biomarkers of developing CSS. This lethal clinical complication is also induced by other viruses (e.g., respiratory syncytial virus and influenza), bacteria, fungi and parasites. CSS usually includes Macrophage Activation Syndrome (MAS) and secondary hemophagocytic lymphohistiocytosis (sHLH), and is characterized by a severe and lethal hypercytokinemia causing multi-organ failure.

As of May 30, 2021, over 170,000,000 people had been infected by the SARS-CoV-2 virus, leading to 3,550,000 deaths and the numbers are growing daily. More than 180 countries have reported laboratory-confirmed cases of COVID-19 on all continents except Antarctica.

In the United States, ˜34 million cases of SARS-COV-2 infection have been confirmed, resulting in more than 600,000 deaths. Since Mar. 26, 2020, the United States has more confirmed infections than any other country in the world.

In the United States (before vaccination), attributable deaths have been most common in adults aged 85 years or older (31%), followed by adults aged 75-84 and 65-74 years (26% and 20%, respectively). The death rate declines by ˜2.5-fold with every decade of age. Worldwide, about 1% of all active cases (confirmed infected patients) are in serious or critical condition.

According to recommendations for COVID-19 patients, adults in septic shock should be administered balanced crystalloid fluids, vasoconstrictor drugs, and corticosteroids in refractory shock and in patients with ARDS. Specifically, COVID-19 patients with SpO₂≤94% on room air, and those who require supplemental oxygen, mechanical ventilation, or extracorporeal membrane oxygenation (ECMO) are advised to be treated with dexamethasone. It is also suggested to use acetaminophen/paracetamol as antipyretic drug for patients with high fever. Similar protocols have been proposed for other infections inducing CSS and septic shock. However, the use of systemic corticosteroids such as dexamethasone in non-critical patients who do not require oxygen supplementation was reported to increase mortality and is contra-indicated, mainly because of the severe suppression of the entire immune system and the risk of elevated viral load in the body. For example, the RECOVERY trial provided evidence that treatment with dexamethasone reduced the 28-day mortality in COVID-19 patients who are receiving respiratory support. Yet, the use of dexamethasone among patients who did not require oxygen had the opposite effect and it significantly increased mortality.

US Publication No. 2019/0160058 describes methods for treating a CB2 receptor-related disorder (e.g., pain, fibrosis). These methods are directed to reducing the risk of adverse events based on reduced blood pressure and/or heart rate in subjects in need of treatment with a CB2 receptor agonist compound.

There is a need for improved compositions for the treatment of subjects that were infected with a pathogen to prevent inflammatory complications and deterioration of the disease and reduce disease mortality, which are suitable for a wide range of patients and can be consumed at an early stage of the infection.

### SUMMARY OF THE INVENTION

The present invention provides compositions comprising at least one selective CB2 receptor agonist and at least one steroid for treating subjects infected with a pathogen and for reducing or even preventing inflammatory complications such as sepsis, cytokine storm syndrome (CSS) and septic shock. In particular, at least one CB2 receptor agonist selected from osteogenic growth peptide (OGP) (SEQ ID NO: 1) or its pentapeptide fragment 5aa-OGP (SEQ ID NO: 2), olorinab, β-caryophyllene (BCP) and lenabasum (formerly JBT-101) is combined herein with at least one steroid selected from dehydroepiandrosterone (DHEA), DHEA-sulfate (DHEAs) and pregnenolone. The combinations are effective in treating a subject infected with pathogen, to prevent deterioration of the disease and reduce mortality.

Advantageously, the compositions of the present invention effectively suppress excessive inflammatory responses such as CSS without weakening the humoral/adaptive immune arm of the immune system which directly attacks the pathogen that causes the infection.

Also, the compositions of the present invention are based, in part, on the unexpected discovery that combinations tested in a mouse model of septic shock were remarkably effective in increasing survival rate of the mice.

According to one aspect, the present invention provides a pharmaceutical composition comprising: at least one selective CB2 receptor agonist selected from the group consisting of: osteogenic growth peptide (OGP) (SEQ ID NO: 1), 5aa-OGP (SEQ ID NO: 2), olorinab, β-caryophyllene (BCP) and lenabasum; and at least one steroid selected from the group consisting of: dehydroepiandrosterone (DHEA), dehydroepiandrosterone-sulfate (DHEAs), and pregnenolone.

According to another aspect, the present invention provides a pharmaceutical composition comprising osteogenic growth peptide (OGP) (SEQ ID NO: 1) or 5aa-OGP (SEQ ID NO: 2), for use in treating a subject infected with a pathogen.

According to another aspect, the present invention provides a pharmaceutical composition comprising olorinab, for use in treating a subject infected with a pathogen.

According to yet another aspect, the present invention provides a pharmaceutical composition comprising β-caryophyllene (BCP), for use in treating a subject infected with a pathogen.

According to another aspect, the present invention provides a pharmaceutical composition comprising lenabasum, for use in treating a subject infected with a pathogen.

According to yet another aspect, the present invention provides a composition for human consumption comprising β-caryophyllene (BCP), in combination with at least one steroid selected from the group consisting of: dehydroepiandrosterone (DHEA), dehydroepiandrosterone-sulfate (DHEAs) and pregnenolone.

Another aspect provides a method of treating inflammation and/or associated complications thereof comprising co-administering to a subject in need thereof: (1) at least one selective CB2 receptor agonist selected from the group consisting of: OGP (SEQ ID NO: 1), 5aa-OGP (SEQ ID NO: 2), olorinab, β-caryophyllene (BCP) and lenabasum; and (2) at least one steroid selected from the group consisting of: dehydroepiandrosterone (DHEA), dehydroepiandrosterone-sulfate (DHEAs) and pregnenolone.

According to yet another aspect, the present invention provides a method of treating a subject infected with a pathogen, the method comprising co-administering to the subject: at least one selective CB2 receptor agonist selected from the group consisting of: OGP (SEQ ID NO: 1), 5aa-OGP (SEQ ID NO: 2), olorinab, β-caryophyllene (BCP) and lenabasum; and at least one steroid selected from the group consisting of: dehydroepiandrosterone (DHEA), dehydroepiandrosterone-sulfate (DHEAs) and pregnenolone.

According to another aspect, the present invention provides a method of treating a subject infected with a pathogen, the method comprising the step of administering to the subject a pharmaceutical composition comprising a selective CB2 receptor agonist selected from the group consisting of: OGP (SEQ ID NO: 1), 5aa-OGP (SEQ ID NO: 2), olorinab, β-caryophyllene (BCP) and lenabasum.

According to some embodiments, a pharmaceutical composition of the present invention comprises: 5aa-OGP (SEQ ID NO: 2) and at least one steroid selected from the group consisting of dehydroepiandrosterone (DHEA), dehydroepiandrosterone-sulfate (DHEAs) and pregnenolone. According to some embodiments, the pharmaceutical composition comprises 5aa-OGP and DHEA. According to some embodiments, the pharmaceutical composition comprises 5aa-OGP and DHEAs. According to some embodiments, the pharmaceutical composition comprises 5aa-OGP and pregnenolone.

According to some embodiments, a pharmaceutical composition of the present invention comprises: OGP (SEQ ID NO: 1) and at least one steroid selected from the group consisting of dehydroepiandrosterone (DHEA), dehydroepiandrosterone-sulfate (DHEAs) and pregnenolone. According to some embodiments, the pharmaceutical composition comprises OGP and DHEA. According to some embodiments, the pharmaceutical composition comprises OGP and DHEAs. According to some embodiments, the pharmaceutical composition comprises OGP and pregnenolone.

According to another aspect, the present invention provides a method of treating a subject infected with a pathogen, the method comprising the step of co-administering a CB2 receptor agonist and a steroid.

According to an additional aspect, the present invention provides a pharmaceutical composition comprising: (1) a peptide comprising an amino acid sequence YGFGG (5aa-OGP; SEQ ID NO: 2), or an analog or derivative thereof, and (2) at least one steroid selected from the group consisting of: dehydroepiandrosterone (DHEA), dehydroepiandrosterone-sulfate (DHEAs) and pregnenolone; and a carrier, diluent or excipient.

According to some embodiments, a pharmaceutical composition of the present invention comprises: olorinab and at least one steroid selected from the group consisting of dehydroepiandrosterone (DHEA), dehydroepiandrosterone-sulfate (DHEAs) and pregnenolone. According to some embodiments, the pharmaceutical composition comprises olorinab and DHEA. According to some embodiments, the pharmaceutical composition comprises olorinab and DHEAs. According to some embodiments, the pharmaceutical composition comprises olorinab and pregnenolone.

According to some embodiments, a pharmaceutical composition of the present invention comprises: β-caryophyllene (BCP) and at least one steroid selected from the group consisting of dehydroepiandrosterone (DHEA), dehydroepiandrosterone-sulfate (DHEAs) and pregnenolone. According to some embodiments, the pharmaceutical composition comprises: BCP and DHEA. According to some embodiments, the pharmaceutical composition comprises: BCP and DHEAs. According to some embodiments, the pharmaceutical composition comprises: BCP and pregnenolone.

According to some embodiments, a pharmaceutical composition of the present invention comprises: lenabasum and at least one steroid selected from the group consisting of dehydroepiandrosterone (DHEA), dehydroepiandrosterone-sulfate (DHEAs) and pregnenolone. According to some embodiments, the pharmaceutical composition comprises: lenabasum and DHEA. According to some embodiments, the pharmaceutical composition comprises: lenabasum and DHEAs. According to some embodiments, the pharmaceutical composition comprises: lenabasum and pregnenolone.

According to some embodiments, the at least one selective CB2 receptor agonist and the at least one steroid are in a single pharmaceutical composition.

According to some embodiments, a pharmaceutical composition of the present invention is for use in treating inflammation and/or associated complications thereof.

According to some embodiments, a pharmaceutical composition of the present invention is for use in treating a subject infected with a pathogen. According to some embodiments, the pathogen is a virus. According to some embodiments, the virus is a coronavirus. According to some embodiments, the corona virus is ß-coronavirus. According to some embodiments, the β-coronavirus is Severe Acute Respiratory Syndrome Coronavirus 2 (SARS-COV-2).

According to some embodiments, a pharmaceutical composition of the present invention is for use in treating a subject that suffers from a respiratory syndrome. According to some embodiments, the respiratory syndrome is selected from the group consisting of viral pneumonia, lung angioedema, pulmonary embolism, severe acute respiratory syndrome, and acute respiratory distress syndrome (ARDS). Each possibility represents a separate embodiment of the present invention.

According to some embodiments, a pharmaceutical composition of the present invention is for use in treating a subject that suffers from sepsis and/or septic shock. According to some embodiments, the subject suffers from cytokine storm.

According to some embodiments, a pharmaceutical composition of the present invention is for use in treating a subject suffers from post-infection symptoms. According to some embodiments, the subject suffers from post-viral syndrome. According to some embodiments, the subject suffers from long-haul COVID-19.

A subject according to the present invention is typically a human subject. In some embodiments, a subject according to the present invention is a non-human mammal.

According to an additional aspect, the present invention provides a method of treating a pathogen infection, inflammation and/or associated complications, comprising administering to a subject in need of such treatment a therapeutically effective amount of: (1) at least one selective CB2 receptor agonist selected from the group consisting of olorinab, β-caryophyllene (BCP), lenabasum, and a peptide comprising an amino acid sequence YGFGG (5aa-OGP; SEQ ID NO: 2), or an analog or derivative thereof; and (2) at least one steroid selected from the group consisting of: dehydroepiandrosterone (DHEA), dehydroepiandrosterone-sulfate (DHEAs), and pregnenolone.

According to some embodiments, the peptide is selected from the group consisting of OGP (SEQ ID NO: 1) and 5aa-OGP (SEQ ID NO: 2).

According to some embodiments, the CB2 receptor agonist and the steroid are co-formulated. According to some embodiments, the CB2 receptor agonist and the steroid are present within a single pharmaceutical composition. According to some embodiments, the CB2 receptor agonist and the steroid are co-formulated in a pharmaceutical composition further comprising at least one carrier, diluent or excipient.

According to some embodiments, the method comprising administering a pharmaceutical composition comprising a CB2 receptor agonist as described herein, and a pharmaceutical composition comprising a steroid as described herein.

According to some embodiments, the administering of the CB2 receptor agonist and the steroid is carried out substantially simultaneously, concurrently, alternately, sequentially or successively. Each possibility represents a separate embodiment of the invention. According to certain embodiments, the administering of the CB2 receptor agonist and the steroid is carried out simultaneously.

According to some embodiments, the pharmaceutical composition(s) is administered at least twice a day, once a day, twice a week or once a week. According to some embodiments, the pharmaceutical composition(s) is administered at least once a day.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B show the survival of septic mice treated with 5aa-OGP (3 different doses, FIG. 1A; optimal dose only, FIG. 1B) versus control (PBS).

FIG. 2 shows the survival of septic mice treated with olorinab (3 different doses) versus control (PBS).

FIGS. 3A-3B show survival of septic mice treated with: DHEA+5aa-OGP versus 5aa-OGP/DHEA/no treatment (FIG. 3A), and DHEA+olorinab versus olorinab/DHEA/no treatment (FIG. 3B).

FIGS. 4A-4D show disease progression and survival of septic mice treated with: BCP or Pregnenolone alone (progression-FIG. 4A, survival-FIG. 4B) or a combination of βCP+Pregnenolone (progression-FIG. 4C, survival-FIG. 4D) versus PBS.

FIGS. 5A-5B show the effect of treating cells with 5aa-OGP vs. CB1/CB2 receptor agonists on cAMP levels. FIG. 5A shows cAMP levels after treatment of CHO cells transfected with human CB2 receptor with: forskolin (FSK), FSK+5aa-OGP (“H4(99-103)”) or FSK+the CB2 receptor agonist HU910; FIG. 5B shows cAMP levels after treatment of HEK293 cells transfected with human CB1 receptor with: forskolin (FSK), FSK+5aa-OGP (“H4(99-103)”) or FSK+the CB1 receptor agonist HU210.

FIGS. 6A-6C show levels of the inflammation markers: TNF-α (FIG. 6A), IL-1β (FIG. 6B) and IL-6 (FIG. 6C), in primary macrophages treated with LPS or LPS+5aa-OGP (“H4(99-103)”).

FIGS. 7A-7B show measurements (“mm”) of ear edema caused by xylene applied to the surface of one ear of WT mice (FIG. 7A) and CB2 receptor-knockout mice (FIG. 7B) after treatment with 5aa-OGP (“H4(99-103)”), HU910, indomethacin (Indo) or vehicle (Veh).

FIGS. 8A-8F show bone marrow (BM, FIGS. 8A-8B) and spleen (FIGS. 8C-8F) T-cells of mice injected with 5aa-OGP (“OGP”), HU910 or vehicle (“VEH”) for 5 days. FIG. 8A—CD4+ in BM. FIG. 8B—CD8+ in BM. FIG. 8C—CD4+CD25+FOXP3+ regulatory T cells (CD4+ Treg cells) in spleen. FIG. 8D—FOXP3 mRNA levels in spleen. FIG. 8E—CD4+ T cells in spleen. FIG. 8F—CD8+ T cells in spleen.

FIG. 9 shows change of IFN expression in septic mice treated with 5aa-OGP (“OGP”), olorinab or saline (LPS only), or untreated (“control”).

### DETAILED DESCRIPTION OF THE INVENTION

Provided herein are compositions comprising at least one selective CB2 receptor agonist and at least one steroid for treating pathogen infected subjects and for reducing inflammatory complications such as sepsis, cytokine storm syndrome (CSS) and septic shock.

The combination of the CB2 receptor agonist and the steroid in some embodiments provides a synergistic effect. As used herein, “synergy” or “synergistic” interchangeably refer to the combined effects of two active agents that are greater than their additive effects. Synergy can also be achieved by producing an efficacious effect with combined inefficacious doses of two active agents.

According to one aspect, the present invention provides a pharmaceutical composition or pharmaceutical combination comprising: at least one selective CB2 receptor agonist selected from the group consisting of: osteogenic growth peptide (OGP) (SEQ ID NO: 1), 5aa-OGP (SEQ ID NO: 2), olorinab, ß-caryophyllene (BCP) and lenabasum; and at least one steroid selected from the group consisting of dehydroepiandrosterone (DHEA), dehydroepiandrosterone-sulfate (DHEAs), and pregnenolone.

According to another aspect, the present invention provides a method of treating a pathogen infection, inflammation, and/or associated complications, comprising administering to a subject in need of such treatment a therapeutically effective amount of: (1) at least one selective CB2 receptor agonist selected from the group consisting of a peptide comprising an amino acid sequence YGFGG (5aa-OGP; SEQ ID NO: 2), or an analog or derivative thereof, olorinab, β-caryophyllene (BCP) and lenabasum; and (2) at least one steroid selected from the group consisting of dehydroepiandrosterone (DHEA), dehydroepiandrosterone-sulfate (DHEAs), and pregnenolone.

### DHEA and DHEA-Sulfate

Dehydroepiandrosterone (DHEA) is an endogenous steroid hormone clinically administered as a therapy for postmenopausal sexual dysfunction (“Intrarosa”), for lupus (off-label use), and other indications. DHEA is also sold as a dietary supplement in many countries. In a septic challenge study in mice, DHEA administration led to an increased survival. The immunoenhancing effect of DHEA was accompanied by a reduction of TNF-α release and an improved activity of T-cellular immunity (Oberbeck et al. “Dehydroepiandrosterone decreases mortality rate and improves cellular immune function during polymicrobial sepsis”. Crit Care Med 2001. 29:380-384).

### Pregnenolone

Pregnenolone (P5), or pregn-5-en-3β-ol-20-one, is a naturally occurring and endogenous steroid. It is a precursor/metabolic intermediate in the biosynthesis of most of the steroid hormones, an anti-inflammatory drug and neurosteroid. In the 1950s it was used in the treatment of rheumatoid arthritis and soft-tissue rheumatism, but is no longer used today.

### CB2 Receptor

Cannabinoid receptor type 2 (CB2) inhibits the activity of adenylyl cyclase through Gi/Goa subunits. CB2 can lead to an increase of intracellular cAMP, previously shown for several types of human leukocytes. Its signaling pathways also include activation of pERK and G protein-coupled Inward Rectifying K⁺-channels (GIRKs) and recruitment of β-arrestin to the receptor (Felder et al. “Comparison of the pharmacology and signal transduction of the human cannabinoid CB1 and CB2 receptors”. Mol. Pharmacol. 1995. 48:443-450; Howlett et al. “International Union of Pharmacology. XXVII. Classification of cannabinoid receptors”. Pharmacol. Rev. 2002. 54:161-202; Dhopeshwarkar & Mackie. “CB2 Cannabinoid receptors as a therapeutic target-what does the future hold?”. Mol. Pharmacol. 2014. 86:430-437). It is currently unknown which signal transduction pathways (or combinations thereof) are relevant for therapeutic purposes. In addition, some compounds may act as biased and/or protean agonists. CB2 receptor is expressed in many tissues and cell types (such as brain, immune system, gastrointestinal system, bone and peripheral nervous system). In the immune cells, CB2 regulates cytokine release. Changes in CB2 receptor expressions and/or endocannabinoid levels were shown in various diseases.

Cannabinoid receptor 2 (CB2) activation is associated with the suppression of inflammation. In macrophages, CB2 stimulation suppresses the release of pro-inflammatory cytokines. It also suppresses neutrophil migration and differentiation, but induces natural killer cell migration and infiltration into inflamed tissues and organs. However, CB2 in not involved in the suppressive effects of T cells proliferation and activation, suggesting CB2 selectively impacts the innate and adaptive immunity. CB2 activation has been suggested to be a potential therapeutic target for sepsis (He et al. “Cannabinoid receptor 2: a potential novel therapeutic target for sepsis?”, Acta Clin Belg. 2019. 74(2):70-74; Lehmann et al. “Cannabinoid receptor 2 activation reduces intestinal leukocyte recruitment and systemic inflammatory mediator release in acute experimental sepsis”. Critical Care 2012. 16:R47). Yet, not all CB2 receptor agonists are selective or potent enough to effectively suppress the cytokine storm without activating CB1, and some may have biased CB2 signaling displaying antagonism/inverse agonism. Some selective CB2 receptor agonists have internalization issues which partially explained by the differences between partial and full CB2 receptor agonists. Decreased number of membranal CB2 receptors may cause diminishing efficacy.

### OGP

Osteogenic growth peptide (OGP) is secreted primarily by osteoblasts, the bone-forming cells, and shares the primary structure of a segment of the C-terminus region of histone H4. In response to bone marrow injury, OGP level is increased in the serum in vivo driving a systemic osteogenic response. It promotes bone cell proliferation and differentiation when administered to pre-osteoblasts in vitro. OGP stimulates osteogenesis and hematopoiesis when administered in vivo. OGP increases bone mass. OGP is a 14-amino acid (14-aa) peptide (a.k.a. OGP[1-14]) resulting from an alternative translation initiation of the histone H4 gene and is therefore also named H4[90-103] in mice and H4[89-102] in humans. This 14-aa peptide is degraded by non-specific enzymes into a 5-amino acid fragment (OGP[10-14] or 5aa-OGP) that shares the CB2 receptor agonism function of the 14-aa peptide OGP. OGP is highly conserved in mammals and present at physiological levels in the serum. 5aa-OGP has exactly the same sequence in most mammals.

A used herein, the terms “osteogenic growth peptide” and “OGP” refer to the 14 aa-long peptide with the sequence set forth as SEQ ID NO: 1 [ALKRQGRTLYGFGG]. The term “5aa-OGP” refers to the pentapeptide corresponding to the last 5 carboxy terminal amino acids of OGP. The sequence of the pentapeptide form (5aa-OGP) is set forth as SEQ ID NO: 2 [YGFGG].

### Olorinab

Olorinab (formerly “APD371”) is an orally available small molecule that is a selective CB2 full agonist (Han et al. “Discovery of APD371: Identification of a Highly Potent and Selective CB2 Agonist for the Treatment of Chronic Pain”. ACS Med Chem Lett. 2017. 8(12):1309-1313). Olorinab completed Phase IIa for treating IBD and recently completed Phase IIb for treating IBS.

### Lenabasum

Lenabasum (formerly “JBT-101”) is an orally available small molecule that preferentially binds to the cannabinoid receptor CB2. In Phase II studies in diffuse cutaneous systemic sclerosis (dcSSc) and patients with dermatomyositis with active skin involvement, the drug demonstrated improvement in multiple physician-assessed and patient-reported efficacy outcomes.

### BCP

β-caryophyllene (BCP or βCP), is a natural bicyclic sesquiterpene that is a constituent of many plants extracts and essential oils. BCP is a phytocannabinoid and a selective agonist of the cannabinoid receptor CB2. BCP was found to have several bioactivities including anti-inflammatory, antibacterial, analgesic, local anesthetic, anti-carcinogenic, antioxidant and lipid-lowering activities. BCP is approved as a food additive by the US Food and Drug Administration (FDA) and the European Food Safety Authority (EFSA) and can be detected in its intact form in serum after oral administration.

According to some embodiments, the pharmaceutical composition comprises osteogenic growth peptide (OGP) (SEQ ID NO: 1) and dehydroepiandrosterone (DHEA). According to some embodiments, the pharmaceutical composition comprises osteogenic growth peptide (OGP) (SEQ ID NO: 1) and dehydroepiandrosterone-sulfate (DHEAs). According to some embodiments, the pharmaceutical composition comprises osteogenic growth peptide (OGP) (SEQ ID NO: 1) and pregnenolone.

According to some embodiments, the pharmaceutical composition comprises 5aa-OGP (SEQ ID NO: 2) and dehydroepiandrosterone (DHEA). According to some embodiments, the pharmaceutical composition comprises 5aa-OGP (SEQ ID NO: 2) and dehydroepiandrosterone-sulfate (DHEAs). According to some embodiments, the pharmaceutical composition comprises 5aa-OGP (SEQ ID NO: 2) and pregnenolone.

According to some embodiments, the pharmaceutical composition comprises olorinab and dehydroepiandrosterone (DHEA). According to some embodiments, the pharmaceutical composition comprises olorinab and dehydroepiandrosterone-sulfate (DHEAs). According to some embodiments, the pharmaceutical composition comprises olorinab and pregnenolone.

According to some embodiments, the pharmaceutical composition comprises β-caryophyllene (BCP) and dehydroepiandrosterone (DHEA). According to some embodiments, the pharmaceutical composition comprises β-caryophyllene (BCP) and dehydroepiandrosterone-sulfate (DHEAs). According to some embodiments, the pharmaceutical composition comprises β-caryophyllene (BCP) and pregnenolone.

According to some embodiments, the pharmaceutical composition comprises β lenabasum and dehydroepiandrosterone (DHEA). According to some embodiments, the pharmaceutical composition comprises lenabasum and dehydroepiandrosterone-sulfate (DHEAs). According to some embodiments, the pharmaceutical composition comprises lenabasum and pregnenolone.

Ingredients of the compositions of the present invention may be artificially synthesized. Alternatively or additionally, ingredients which are found in natural sources, such as plant sources, may be extracted or purified from the natural sources. The present invention encompasses artificially synthesized compounds as well as compounds extracted and/or purified from natural sources.

The term “pharmaceutical composition” as used herein refers to any composition comprising at least one pharmaceutically active ingredient, formulated such that it facilitates accessibility of the active ingredient to the target organ. The term “pharmaceutical composition” further includes a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” as used herein indicates an inactive substance that serves as mechanisms to improve the delivery and the effectiveness of drugs and can be identified by a skilled person in view of the route of administration and related composition formulation.

The term “excipient” as used herein indicates an inactive substance that can be used any of various media acting usually as coloring agents, preservatives, coatings, solvents, binders or diluents to bulk up formulations that contain active ingredients (thus often referred to as “bulking agents.” “fillers.” or “diluents”), to allow convenient and accurate dispensation of a drug substance when producing a dosage form. Suitable excipients can include any substance that can be used to bulk up formulations with the pentapeptide described herein or CBD to allow for convenient and accurate dosage.

Compositions according to the present invention may be in a form selected from the group consisting of a pill, a tablet, a caplet, a capsule, a softgel, a powder, and a lozenge. Additional suitable forms include sublingual and/or buccal administration compositions, sachets, bars, gummies, gummy caps, in yogurt and in functional foods and functional drinks. Compositions according to the present invention may be formulated for administration as a nasal spray. Compositions according to the present invention may also be formulated for administration via routs such as intravenous (IV), subcutaneous (SC), intramuscular (IM) and intraperitoneal (IP), or using inhalers (including disks) and inhalation vaporizers. Each of the aforementioned possibilities represents a separate embodiment of the present invention. According to certain embodiments, the pharmaceutical composition is administered orally.

According to some embodiments, the pharmaceutical composition or combination is formulated in a form of a liquid or a gel. According to other embodiments, the pharmaceutical composition or combination is a non-aqueous composition.

According to some embodiments, the pharmaceutical composition or combination is formulated as a capsule, a tablet, a liquid, or a syrup. In certain embodiments, the dosage form is granules or pellets delivered in a sachet or filled into capsule or compressed into a tablet.

According to some embodiments, the pharmaceutical composition or combination further comprises triglycerides, fats, lipids, oils, fatty acids, co-solvents or mixtures thereof. According to certain embodiments, the pharmaceutical composition or combination comprises an edible oil or fat.

According to some embodiments, the pharmaceutical composition is formulated for slow release of the active component. Orally administered formulations such as tablets may optionally be coated or scored and may be formulated so as to provide sustained, delayed or controlled release of the active ingredient therein.

The terms “treatment” and “treating”, used herein interchangeably, refer to the diminishment, alleviation, or amelioration in the number, severity, or frequency of at least one clinical symptom or biochemical indices associated with or caused pathogen infection and related inflammatory complications.

The term “pathogen” as used herein refers to an infectious biological agent or to proteinaceous infectious particle (prion) that causes a disease state in a host. Pathogens include a virus, bacterium, fungus, protozoan, parasite and prion. Each possibility represents a separate embodiment. In some particular embodiments, the pathogen is a virus. In additional particular embodiments, the pathogen is a bacterium.

The term “infected with a pathogen” as used herein refers to a subject currently having an infection, namely, the pathogen can be detected in the subject's body, as well as a subject in which the pathogen is no longer detected but symptoms still persist.

The term “post-infection symptoms” as used herein refers to a condition where symptoms associated with an infection, such as fatigue or weakness, linger for weeks to months, even after the infection is no longer detectable in the body. The term “post-viral syndrome” or “post-viral fatigue” refer to post-infection symptoms wherein the infection was caused by a virus. The symptoms of these conditions include but are not limited to: headaches, fatigue, weakness, brain fog/trouble concentrating, joint stiffness, muscle pain, memory/concentration problems, sleep abnormalities, recurring symptoms of viral infections, such as sore throat, headache and swollen lymph nodes.

The term “long-haul COVID-19”, also known as “Post-COVID Conditions”, “Post-Acute Sequelae of SARS-COV-2 infection (PASC)” and “Post-Acute Sequelae of COVID-19 (PASC)”, as used herein, refers to post-viral syndrome caused post infection with SARS-COV-2, wherein a subject experiences new, recurring or ongoing symptoms more than 4 weeks after infection, even if the virus is no longer detected in the body. The symptoms may persist from weeks to months, for example, over 60 days, up to 6 months, and even more than 6 months.

Symptoms are varied and include different combinations of: tiredness or fatigue, difficulty thinking or concentrating (sometimes referred to as “brain fog”), headache, loss of smell or taste, dizziness on standing, fast-beating or pounding heart (also known as heart palpitations), chest pain, difficulty breathing or shortness of breath, cough, joint or muscle pain, depression or anxiety, fever, symptoms that get worse after physical or mental activities. Long-haul COVID-19 symptoms are listed for example in the CDC website: www.cdc.gov.

The term “complications associated with pathogen infections”, particularly inflammatory complication, as used herein, comprise, but not limited to: macrophage activation syndrome (MAS), multisystem inflammatory syndrome in adults (MIS-A), multisystem inflammatory syndrome in children (MIS-C), sepsis, cytokine storm syndrome (CSS), and septic shock.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

As used herein, the term “subject” designates a mammal, preferably a human.

According to an additional aspect, the present invention provides a pharmaceutical composition comprising: (1) a peptide comprising an amino acid sequence YGFGG (5aa-OGP; SEQ ID NO: 1), or an analog or derivative thereof, and (2) at least one steroid selected from the group consisting of: dehydroepiandrosterone (DHEA), dehydroepiandrosterone-sulfate (DHEAs) and pregnenolone; and a carrier, diluent or excipient.

According to some embodiments, the peptide length is up to 50 amino acids residues. According to certain embodiments, the peptide length is up to 45, 40, 35, 30, 25, or 20 amino acid residues. Each possibility represents a separate embodiment of the invention.

According to some embodiments, the peptide is a synthetic peptide.

The term “amino acid” refers to any one of the proteinogenic amino acids, including the 20 genetically-encoded amino acids, biosynthetically available amino acids which are not found in proteins (e.g., 4-hydroxy-proline, 5-hydroxy-lysine, citrulline, ornithine, canavanine, djenkolic acid, β-cyanoalanine, GABA, L-DOPA, T3), and also non-natural and/or amino acids that have been chemically modified (synthetic), each amino acid being characterized by having an amino and a carboxy terminus. The amino acids used in this invention are those which are available commercially or are available by routine synthetic methods. The amino acids are represented throughout the specification and claims by either one or three-letter codes, as is commonly known in the art. When there is no indication, the L stereoisomer was used. The D isomers are indicated by “D” or “(D)” before the residue abbreviation.

The term “amino acid residue” means the moiety which remains after the amino acid has been conjugated to additional amino acid(s) to form a peptide, or to a moiety (such as a permeability-enhancing moiety), typically through the alpha-amino and carboxyl of the amino acid.

The term “peptide” refers to a short chain of amino acids, whether natural, synthetic or recombinant.

The term “synthetic peptides” refers to artificial, non-naturally occurring peptides, typically produced by standard peptide synthesis methods known in the art such as solid-phase peptide synthesis (SPPS). In some embodiments, recombinant protein techniques, well known in the art, are used to generate the peptides of the present invention.

According to some embodiments, the peptide is of 5-14 amino acids, 5-13 amino acids, 5-12 amino acids, 5-11 amino acids, 5-10 amino acids, 5-9 amino acids, 5-8 amino acids, 5-7 amino acids, 5-6 amino acids, 5 amino acids, or 14 amino acids. Each possibility represents a separate embodiment of the invention.

Peptides according to the present invention may include chemically modified amino acids. The term “chemically modified”, when referring to an amino acid, refers to an amino acid that is modified by one or more chemical modifications, which can be performed by techniques known in the art. Chemical modifications of amino acids encompass, but not limited to, acetylation, acylation, amidation, ADP-ribosylation, glycosylation, glycosaminoglycanation, methylation (e.g. N-methylation), myristoylation, pegylation, prenylation, phosphorylation, ubiquitination, and the like.

Analogs of the peptides are also within the scope of the present invention. As used herein, “analogs” are peptides which have the amino acid sequence according to the invention except for one or more amino acid changes, typically, conservative amino acid substitutions. In some embodiments, an analog has at least about 75% identity to the sequence of the peptide of the invention, for example at least about 80%, at least about 85%, at least about 90%, at least about 95%, at least about 99% identity to the sequence of the peptide of the invention. Each possibility represents a separate embodiment of the present invention. Analogs are included in the invention as long as they remain pharmaceutically acceptable and their CB2 agonism activity is not severely damaged.

Conservative substitutions of amino acids as known to those skilled in the art are within the scope of the present invention. Conservative amino acid substitutions include replacement of one amino acid with another having the same type of functional group or side chain e.g., aliphatic, aromatic, positively charged, negatively charged. Conservative substitution tables providing functionally similar amino acids are well known in the art.

The following is an example of classification of the amino acids into six groups, each contains amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K), Histidine (H);
5) Isoleucine (I), Leucine (L), Methionine (M), Valine (V); and
6) Phenylalanine (F), Tyrosine (Y), Tryptophan (W).

Analogs according to the present invention may comprise also peptidomimetics. “Peptidomimetics” refers to synthetic molecules containing non-peptidic structural elements that are capable of mimicking the biological action(s) of a natural parent peptide.

Analogs according to the present invention also encompasses peptides in which one or more amino acids has been removed from the sequence, for example, one, two, three, four, five amino acids have been removed. Analogs in which one or more amino acids have been removed from the sequence are included in the invention as long as they remain pharmaceutically acceptable and their CB2 agonism activity is not severely damaged.

Derivatives of the peptides of the invention are also within the scope of the present invention. As used herein, the term “derivatives” cover inter alia derivatives which may be prepared from the functional groups which occur as side chains on the residues or the N- or C-terminal groups, by means known in the art, and are included in the invention as long as they remain pharmaceutically acceptable, i.e., they do not destroy the pharmacological/biological activity of the peptide, and do not confer significant toxic properties on compositions containing it. These derivatives may include, for example, aliphatic esters of the carboxyl groups, amides of the carboxyl groups produced by reaction with ammonia or with primary or secondary amines, N-acyl derivatives of free amino groups of the amino acid residues, e.g., N-acetyl, formed by reaction with acyl moieties (e.g., alkanoyl or carbocyclic aroyl groups), or O-acyl derivatives of free hydroxyl group (e.g., that of seryl or threonyl residues) formed by reaction with acyl moieties.

The peptides of the present invention are typically utilized in a linear form, although it will be appreciated that in cases where cyclization does not severely interfere with peptide characteristics, cyclic forms of the peptide can also be utilized. Cyclization of peptides may take place by any means known in the art, for example through free amino and carboxylic groups present in the peptide sequence, or through amino acids or moieties added for cyclization. Non-limiting examples of cyclization types are: side chain to side chain cyclization, C-to-N terminal cyclization, side chain to terminal cyclization, and any type of backbone cyclization incorporating at least one N₁-ω-substituted amino acid residue/s as described for example in WO 95/33765. Examples of cyclization and analogs of OGP are descried in U.S. Pat. No. 6,479,460.

According to an additional aspect, the present invention provides a pharmaceutical combination comprising: (1) at least one selective CB2 receptor agonist selected from the group consisting of: osteogenic growth peptide (OGP) (SEQ ID NO: 1), 5aa-OGP (SEQ ID NO: 2), olorinab, β-caryophyllene (BCP) and lenabasum; and (2) at least one steroid selected from the group p consisting of: dehydroepiandrosterone (DHEA), dehydroepiandrosterone-sulfate (DHEAs), and pregnenolone.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient.

According to some embodiments, the terms “pharmaceutical composition” and “pharmaceutical combination” are used herein interchangeably.

According to some embodiments, the pharmaceutical composition or combination is for use in treating inflammation and/or associated complications thereof.

According to some embodiments, a pharmaceutical composition or combination is for use in treating a subject infected with a pathogen. According to some embodiments, the pathogen is a virus. According to some embodiments, the virus is a coronavirus. According to some embodiments, the corona virus is β-coronavirus. According to some embodiments, the β-coronavirus is Severe Acute Respiratory Syndrome Coronavirus 2 (SARS-COV-2).

According to some embodiments, a pharmaceutical composition or combination is for use in treating a subject that suffers from a respiratory syndrome. According to some embodiments, the respiratory syndrome is selected from the group consisting of viral pneumonia, lung angioedema, pulmonary embolism, severe acute respiratory syndrome, and acute respiratory distress syndrome (ARDS). Each possibility represents a separate embodiment of the present invention.

According to some embodiments, a pharmaceutical composition or combination is for use in treating a subject that suffers from sepsis and/or septic shock. According to some embodiments, the subject suffers from cytokine storm.

According to some embodiments, the CB2 receptor agonist and the steroid are present in the same pharmaceutical composition. According to some embodiments, the CB2 receptor agonist and the steroid are present in separate pharmaceutical compositions.

The invention also provides a pharmaceutical pack or kit comprising one or more containers filled with one or more of the pharmaceutical compositions of the invention.

It shall be understood that the amount of any active agent that is administered to a patient to treat that patient, will be administered in a therapeutically effective amount, as determined by ordinarily skilled physicians, pharmacologists, and toxicologists, that take into account the weight and age of the patient. In any event, where the drug has been approved by a regulatory authority (e.g., the U.S. Food and Drug Administration), a therapeutically effective amount of the active agent is an amount approved by the regulatory authority.

According to some embodiments, the pharmaceutical composition further comprises an additional therapeutic agent.

According to some embodiments, the pharmaceutical composition comprises a CB2 receptor agonist as described herein in a dosage ranging from 0.5 μg and 100 mg. According to additional embodiments, the pharmaceutical composition comprises a CB2 receptor agonist as described herein in a dosage ranging from about 5 μg to about 50 mg. According to additional embodiments, the pharmaceutical composition comprises a CB2 receptor agonist as described herein in a dosage ranging from about 20 μg to about 30 mg According to additional embodiments, the pharmaceutical composition comprises a CB2 receptor agonist as described herein in a dosage ranging from about 5 μg to about 10 mg.

According to some embodiments, the pharmaceutical composition comprises a OPG or 5aa-OGP peptide as described herein in a dosage ranging from 0.5 and 500 μg. According to additional embodiments, the pharmaceutical composition comprises a CB2 receptor agonist as described herein in a dosage ranging from about 5 to about 250 μg.

According to some embodiments, the method comprises administering the pharmaceutical composition at a daily dose comprising from about 0.5 μg/day to about 100 mg/day of a CB2 receptor agonist as described herein.

According to some embodiments, the pharmaceutical composition comprises a lenabasum as described herein in a dosage ranging from 500 μg to 100 mg. According to some embodiments, the pharmaceutical composition comprises a lenabasum as described herein in a dosage ranging from 2 mg to 50 mg.

According to some embodiments, the pharmaceutical composition comprises a ß-caryophyllene as described herein in a dosage ranging from 500 μg to 100 mg. According to some embodiments, the pharmaceutical composition comprises a ß-caryophyllene as described herein in a dosage ranging from 2 mg to 50 mg.

According to some embodiments, the pharmaceutical composition comprises a steroid as described herein in a dosage ranging from 25 μg and 1 g.

According to some embodiments, the pharmaceutical composition comprises a steroid as described herein in a dosage ranging from 50 μg and 500 mg. According to additional embodiments, the pharmaceutical composition comprises a steroid as described herein in a dosage ranging from about 200 μg to about 100 mg. According to additional embodiments, the pharmaceutical composition comprises a steroid as described herein in a dosage ranging from about 500 μg to about 50 mg.

According to some embodiments, the pharmaceutical composition comprises a pregnenolone as described herein in a dosage ranging from 5 mg to 1 g. According to some embodiments, the pharmaceutical composition comprises a pregnenolone as described herein in a dosage ranging from 10 mg to 500 mg.

According to some embodiments, the pharmaceutical composition is administered twice a day, once a day, twice a week, once a week, once in two weeks, once in three weeks or once a month. According to some embodiments, the pharmaceutical composition is administered for a period of greater than a week. According to some embodiments, the pharmaceutical composition is administered for a period of greater than four weeks.

According to some embodiments, the unit dosage form is administered with food at any time of the day, without food at any time of the day, with food after an overnight fast (e.g., with breakfast).

The singular forms “a”, “an”, and “the” include plural referents unless the context clearly dictates otherwise. Thus, for example, reference to “a pentapeptide” includes one or more of such peptides and equivalents thereof known to those skilled in the art, and so forth.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Male wild type 8-10 weeks old mice of inbred strain C57BL/6J-RCC were obtained from Envigo Ltd. All experiments were in accordance and with the approval of the institutional animal care and use committee of Tel-Aviv University for these experiments (permit number 01-20-022). The mice were divided into treatment groups 24 hours before LPS administration. All treatments were administered intraperitoneally (IP). OGP was administered at the indicated doses only twice, 24 hrs before LPS and right before the LPS at a dose of 0.5, 5 or 15 μg/kg. Olorinab was administered at 0.33, 1, or 5 mg/kg twice a day starting 24 hours before LPS. DHEA was injected once a day at a dose of 30 mg/kg, starting 24 hr before LPS injection. β-Caryophyllene (BCP or BCP, used herein interchangeably) and Pregnenolone were injected every 12 hours starting 24 h before the LPS injection. A single IP injection of LPS was administered at 25 μg/gr dose. This study had 2 control groups as indicated in the experiments below. One that received PBS instead of the LPS injection (‘PBS’), and one that received PBS instead of the treatments (CB2 agonist or steroid) before the LPS injection (‘LPS’). For humane reasons murine sepsis score (MSS) was used as a surrogate for survival (Mai et al. Intensive Care Med Exp, 2018 Jul. 27; 6(1):20. doi: 10.1186/s40635-018-0184-3). A low MSS score (0-2) served as a surrogate for survival while mice that reached the score 3 were defined as critically ill and were euthanized.

Statistical analyses—For survival experiments, Gehan-Breslow-Wilcoxon test was used for multiple groups comparison. For disease progression Dunnett's multiple comparisons test was used. Differences between groups were considered significant when P<0.05.

### Example 1: 5Aa-OGP Increases Survival in a Mouse Model of Septic Shock

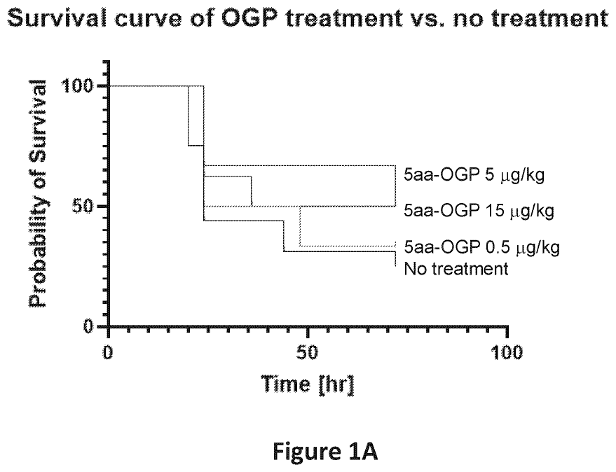

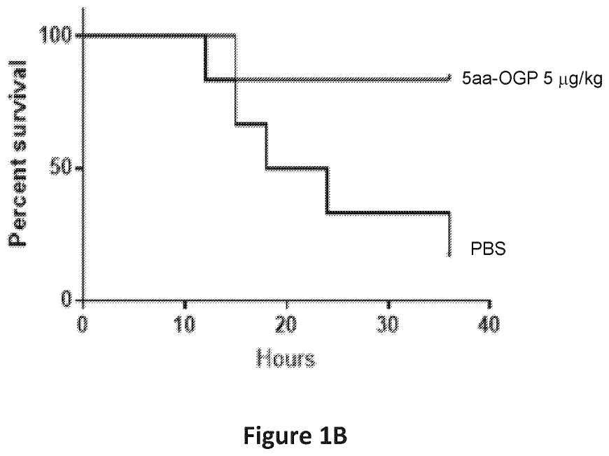

A dose response experiment was conducted to test the potential effect of 5aa-OGP in the sepsis model (FIG. 1A). A single injection of 5aa-OGP was administered i.p. at 3 different doses, 0.5 μg/kg, 5 μg/kg and 15 μg/kg, 24 hours prior to the LPS injection; these concentrations were chosen based on the previous works in the lab.

FIG. 1B demonstrates a trend for increased survival in the 5aa-OGP-treated mice, although these results were not statistically significant probably due to small sample size. The highest increase in survival rates was observed using 5 μg/kg which increased survival by 3.5-fold relative to the LPS group. The highest dose of 15 μg/kg had no noticeable advantage over the medium dose of 5 μg/kg. This 5 μg/kg dose was therefore used in the following experiments.

In a separate experiment, mice received a single i.p. injection of the optimal dose of 5aa-OGP (5 μg/kg) and 24 hours later were intraperitoneal (i.p) injected with a lethal dose of LPS. Control mice received a single PBS injection 24 hours prior to LPS injection. FIG. 1B demonstrates that a single injection of 5aa-OGP significantly increased the survival of LPS-administered mice by 5-fold compared to PBS-treated mice (83.3% vs. 16.6% after 36 hours, respectively) (p=0.03, n=6).

### Example 2: Olorinab Increases Survival in a Mouse Model of Septic Shock

In order to test the therapeutic potential of another CB2 agonist, olorinab was tested in improving survival rate in a mouse model of septic shock induced by LPS injection. Olorinab was administered i.p. 24 hours prior to LPS injection followed by twice-daily injections of olorinab.

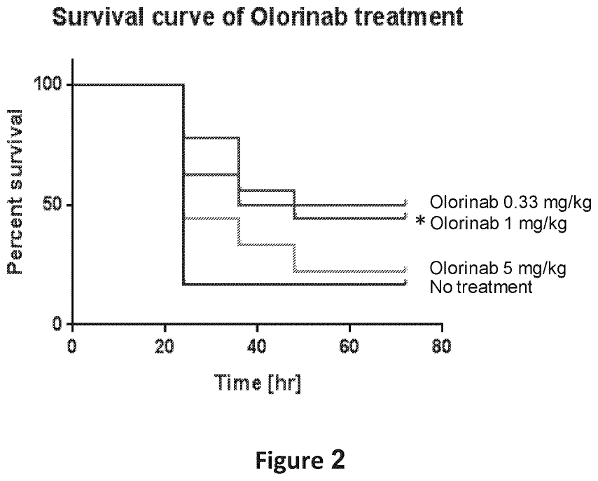

FIG. 2 shows the beneficial effect of different olorinab doses on survival of LPS treated mice, with 1 mg/kg (p<0.05) and 0.33 mg/kg olorinab (administered twice daily, i.p.) having the best survival percentage.

### Example 3: Combinations of DHEA with Olorinab or 5Aa-OGP Increase Survival in a Mouse Model of Septic Shock

The combinations DHEA+olorinab and DHEA+5aa-OGP were tested versus each compound alone and versus vehicle only (no treatment) in LPS-injected mice. The mice received the first dose of treatment 24 hr before LPS injection in the treated groups. All the injections were given i.p., olorinab doses were administered twice a day, DHEA and 5aa-OGP were administered once a day. LPS was given only once.

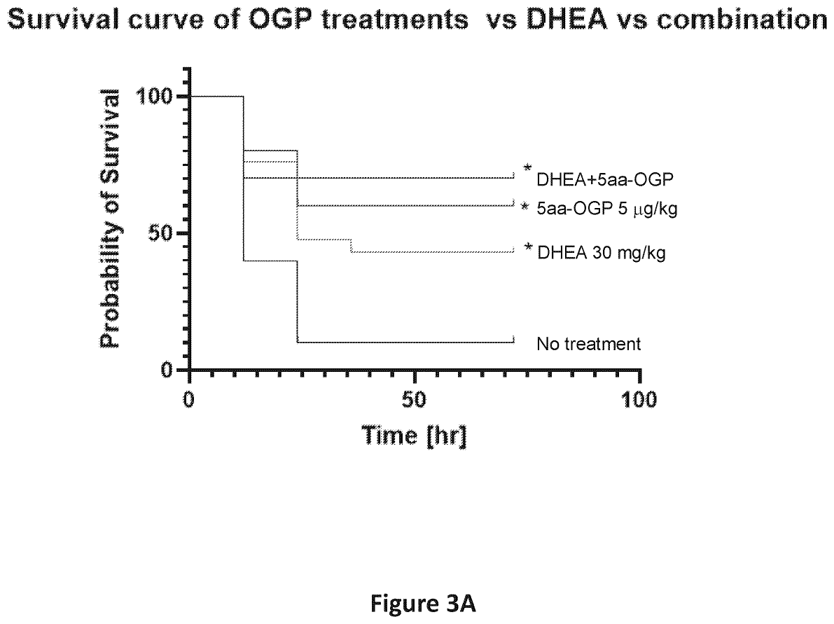

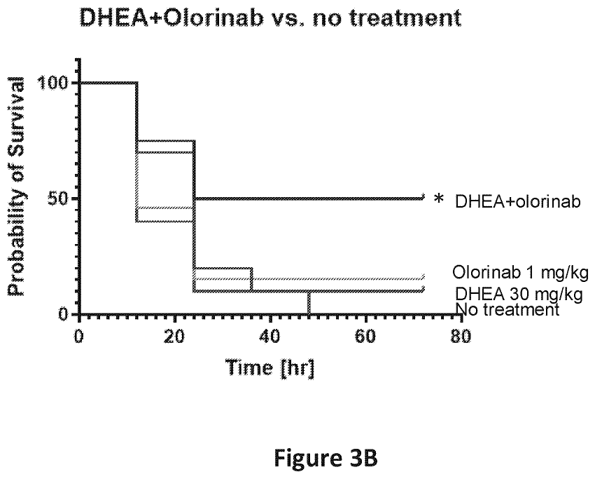

FIG. 3A shows a beneficial effect of combining DHEA with 5aa-OGP on survival compared to each compound alone. FIG. 3B depicts a beneficial effect of combining DHEA with olorinab on survival, compared to each compound alone (synergistic effect).

### Example 4: Combination of BCP with Pregnenolone Increases Survival in a Mouse Model of Septic Shock

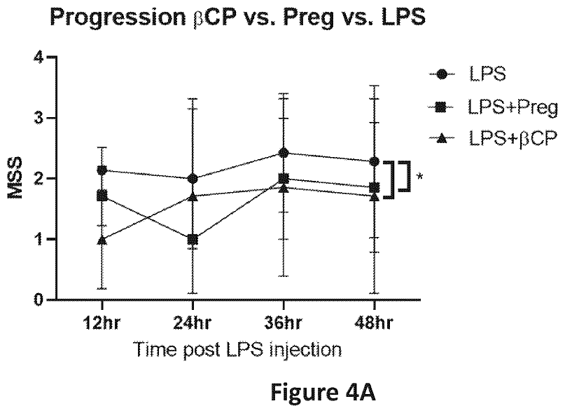

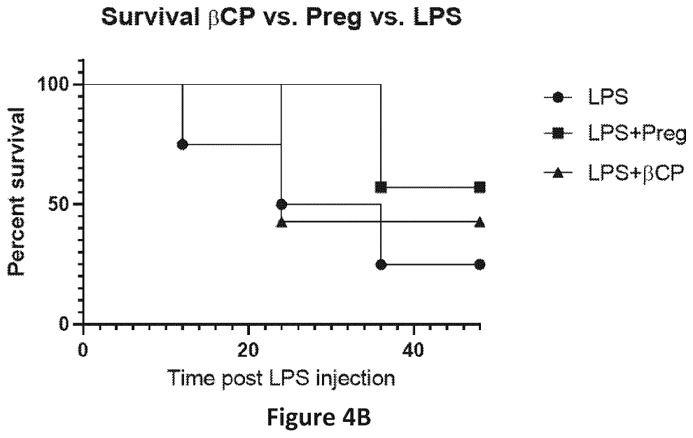

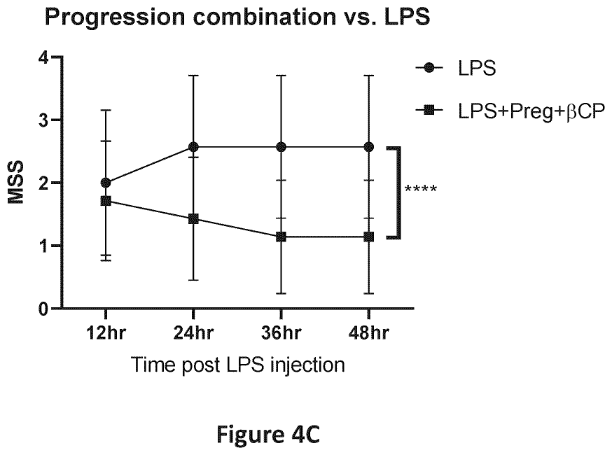

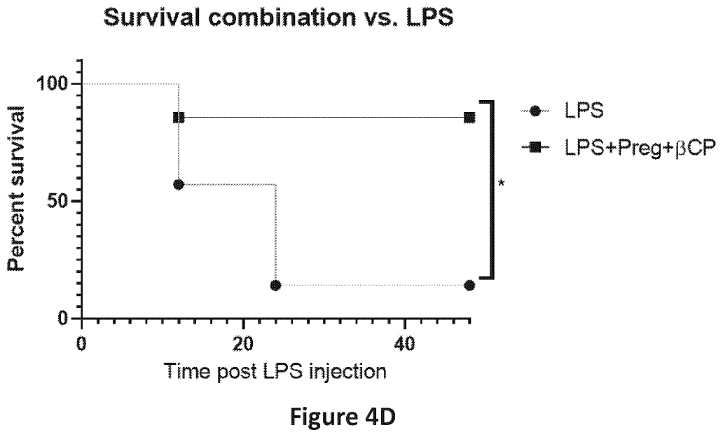

The combination BCP+Pregnenolone was tested versus each compound alone and versus vehicle only (no treatment) in LPS-injected mice. Mice were pre-treated with ß-Caryophyllene (BCP. 25 mg/kg) and pregnenolone (Preg, 10 mg/kg), separately (FIG. 4A) and in combination 24 and 12 hrs before LPS injection (25 mg/kg) and then every 12 hrs. All the injections were given i.p., twice a day. LPS was given only once. A Murine Septic Score (MSS) was recorded every 12 hours (FIGS. 4A and 4C). Survival (FIGS. 4B and 4D) was defined as MSS<3. * P<0.05, n=7.

FIGS. 4A-4B show a slight beneficial effect of BCP in improving disease progression, while Pregnenolone alone had no significant effect (FIG. 4A). These compounds had no significant effect on survival when administered alone (FIG. 4B). FIGS. 4C-4D depict a significant beneficial effect of combining βCP+Pregnenolone on both disease progression and survival (p=0.026), demonstrating a synergistic effect.

### Example 5: Pentapeptide 5Aa-OGP is a Selective CB2 Receptor Agonist

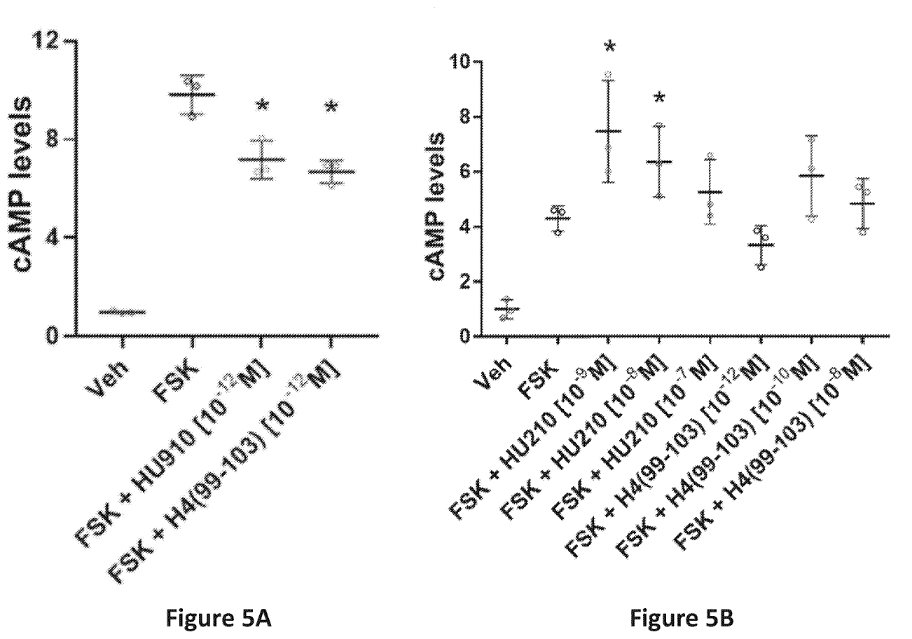

Both CB1 and CB2 negatively regulate adenylate cyclase activity through a pertussis toxin sensitive GTP-binding protein. An agonist of the receptors is expected to reduce the amount of cAMP. A forskolin (FSK) is a diterpene that elevates the level of CAMP in cells. A forskolin assay may be used to test CB1/CB2 receptor agonists activity. CHO cells transfected with human CB2 receptor were treated with FSK, FSK+ 5aa-OGP or FSK+ the CB2 receptor agonist HU910. Control cells were treated with a vehicle. FIG. 5A shows significant reversal of forskolin-stimulated cAMP levels by 5aa-OGP (“H4(99-103)” in the figure), indicating that it is a human CB2 receptor agonist. Data are mean±SD. * p<0.05 vs. FSK, n=3, ANOVA.

HEK293 transfected with human CB1 receptor were treated with FSK, FSK+ 5aa-OGP or FSK+the CB1 receptor agonist HU210 at varying doses. Control cells were treated with a vehicle. FIG. 5B shows no significant increase of FSK-stimulated cAMP levels by 5aa-OGP (“H4(99-103)” in the figure), indicating that it is not a CB1 receptor agonist, and is therefore selective to CB2 receptor. Data are mean±SD. * p<0.05 vs. FSK, n=3, ANOVA.

### Example 6: 5Aa-OGP Inhibits Inflammatory Responses In Vitro and In Vivo

Primary macrophages were treated with LPS or LPS+5aa-OGP at varying doses. Control cells were treated with a vehicle. The level of the pro-inflammatory cytokines TNFα, IL-1β and IL-6 was measured.

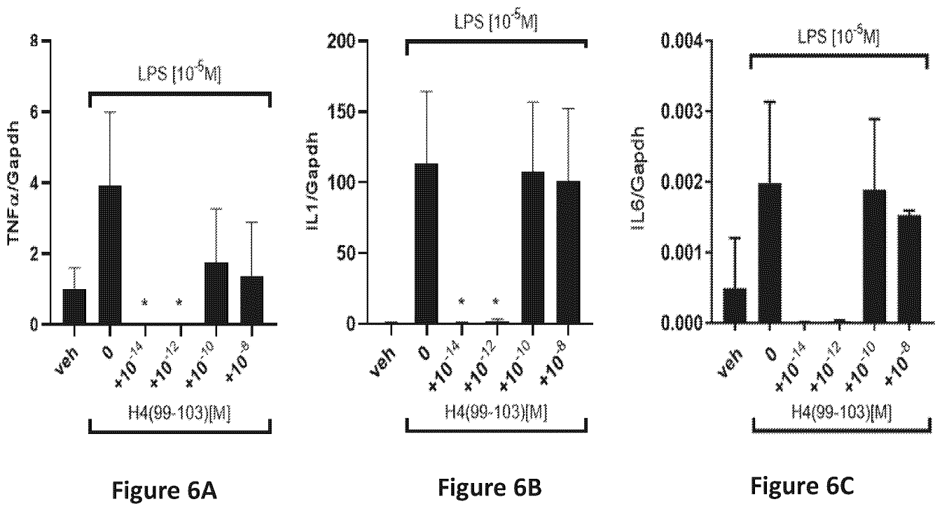

FIGS. 6A-6C show that 5aa-OGP (“H4(99-103)” in the figure) significantly reduces the expression levels of the pro-inflammatory cytokines from activated macrophages in response to LPS in vitro (FIG. 5A-TNF-α, FIG. 5B-IL-1β, FIG. 5C-IL-6). Data are mean±SD, n=3 biological replicates. * p<0.05 vs. LPS (no 5aa-OGP).

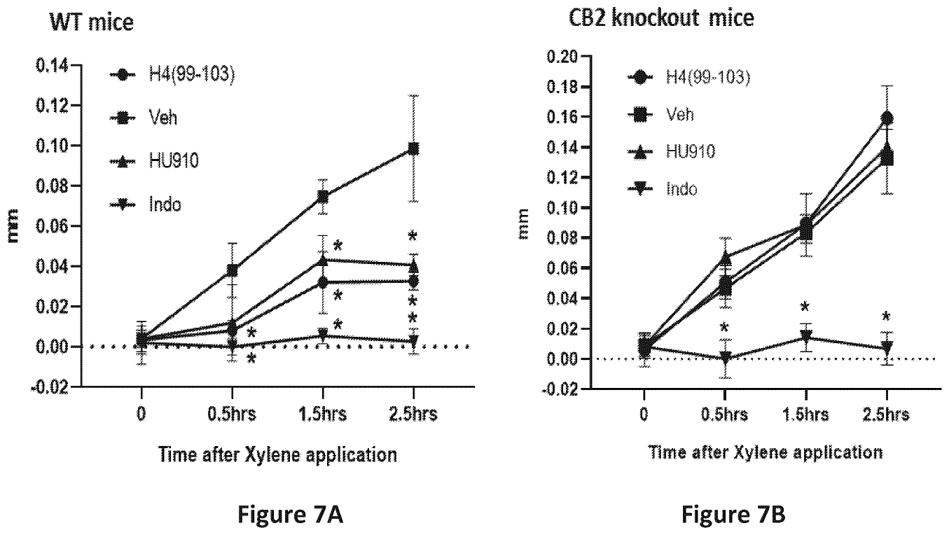

In a further experiment (FIG. 7), xylene was applied to the surface of one ear of WT mice and CB2 receptor-knockout mice to induce edema. 5aa-OGP and HU910 were administered subcutaneously once, 24 hours prior to xylene application, at 10 ng and 20 μg per mouse, respectively. A further group of mice was administered with indomethacin IP, 30 minutes before xylene application. A control group of mice was treated with a vehicle. Ear swelling was measured as difference in ear thickness relative to T0. FIG. 7A shows that 5aa-OGP administration (“H4(99-103)” in the figure) significantly reduces ear edema in WT mice, but has no beneficial effect in CB2 receptor-knockout mice (FIG. 7B). (Mean±SE, n=6. * p<0.05 vs. vehicle (VEH) treated mice). This effect mimics that of HU910, a selective CB2 receptor agonist, further strengthening the conclusion that OGP is a selective CB2 receptor agonist.

### Example 7: 5Aa-OGP does not Affect T-Cells In Vivo

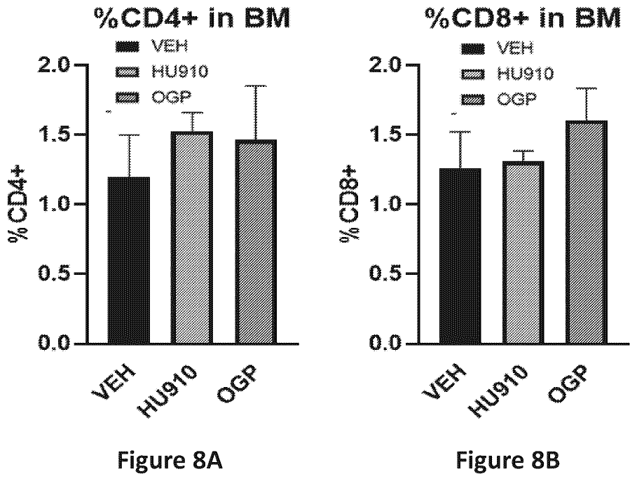

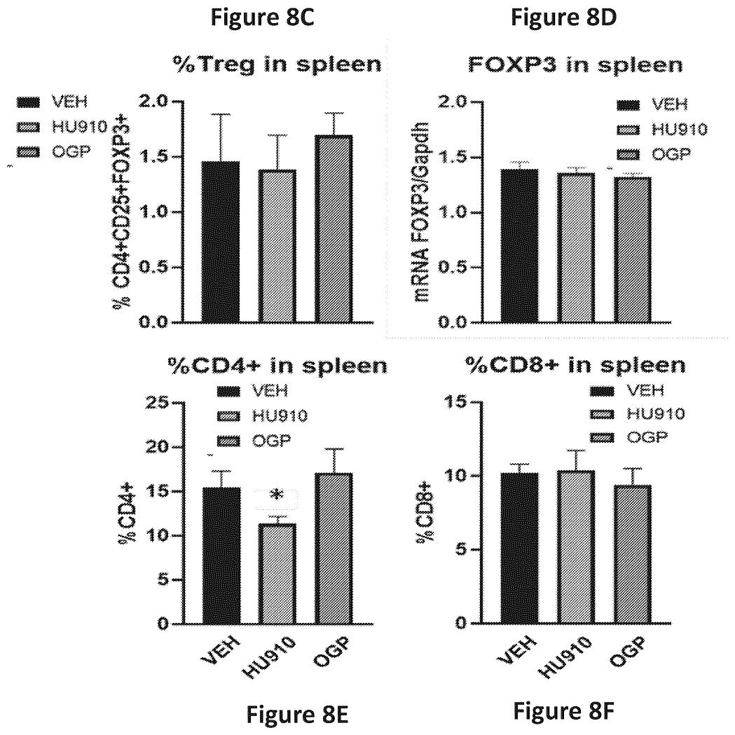

Dexamethasone has been proposed for the treatment of septic shock but preclinical and clinical data show that this treatment modality often results in increased mortality. This is likely due to its strong immunosuppressive actions, including a severe weakening of the humoral/acquired immune system. Our proposed approach uses CB2 agonists that have immunomodulatory roles. On one hand, OGP strongly suppresses the innate immune system, exemplified here as inhibition of cytokine expression by activated macrophages (FIG. 6), and suppression of xylene-induced ear edema (FIG. 7). On the other hand, OGP has no suppressive effects on the adaptive/acquired system, exemplified here by the unaffected number of T cell subsets in mice treated with 5aa-OGP (FIG. 7). 12-week-old mice were injected with 5 μg/kg/day 5aa-OGP, 3 mg/kg/day HU910, or with a vehicle control daily for 5 days. The spleen and bone marrow (BM) were analyzed. FIGS. 8A-8F show that administration of 5aa-OGP (“OGP” in the figure) did not affect T-cells.

### Example 8: 5Aa-OGP Prevents the LPS-Induced Decrease in IFNγ Expression Levels

Interferons (IFN) enhance the immune system in several ways, by exhibiting various biological functions including antiviral, antiproliferative, immunomodulatory and developmental activities (Wang et al. Semin. Immunol. 2019; 43). IFNγ plays an important role in defense against intracellular pathogens such as mycobacteria and viruses. A reduced capacity to produce this cytokine contributes to disease susceptibility (Ouyang et al. Eur Cytok Netw 2002; 13(4):392-4). Mice were injected with 5aa-OGP, olorinab or saline 24 h and 12 h before LPS and RNA was extracted from the lung 12 hours after LPS injection. Control mice were untreated (no LPS). IFNγ expression in lung tissues was analyzed by real-time qPCR.

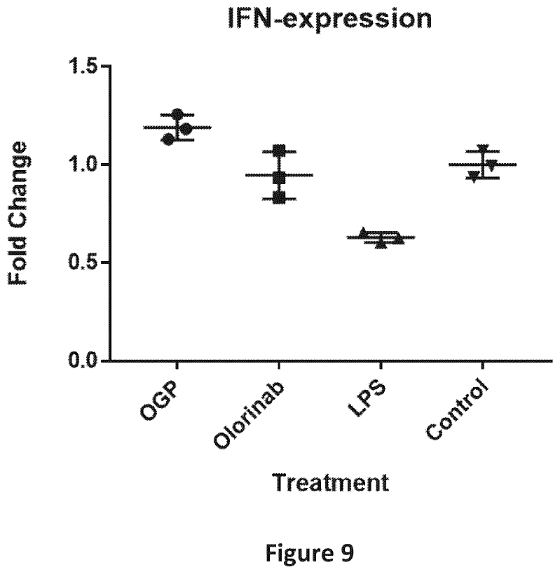

FIG. 9 shows that 5aa-OGP (“OGP” in the figure) not only did not suppress IFNγ expression but increased its expression beyond the levels of the control mice. This indicates that OGP may have positive effects on the immune response to viruses and other pathogens.

### Example 9: Effect of Combination Treatments on Serologic, Cellular and Tissue Parameters in a Septic Shock Mouse Model

C57BL/6J mice are pre-treated with DHEA+olorinab, DHEA+5aa-OGP for 1/0.5 hour prior to LPS injection. The mice are sacrificed 16 hours after LPS injection. Serum, splenocytes and peritoneal cells are collected (for flow cytometry), as well as the liver and the lungs for histological processing. In the serum, cytokine levels (IL-6, TNFα, IL-12, monocyte chemoattractant protein (MCP)-1, IL-1β. IFNγ (proinflammatory), and IL-10 (anti-inflammatory)), as well as HMGB1 and nucleosome (late-phase mediators of sepsis) are measured by ELISA. mRNA of the aforementioned cytokines is measured in peritoneal cells using RT-qPCR. FACS analysis is performed to assess the expression of CD86 and CD40 in splenic F4/80+ macrophages and other immune cells in the lungs. The degree of lung injury is scored based on hemorrhage, lung edema, inflammatory cell infiltration, hyaline membrane, and atelectasis. An in situ TUNEL assay is performed to detect apoptotic pneumonocytes in the lungs.

### Example 10: Comparing Individual Compounds and Combinations to Dexamethasone in a Mouse Model of Septic Shock

The only treatment modality currently approved for the management of the cytokine storm in critical COVID-19 patients is Dexamethasone. LPS-induced septic shock model as described in the previous examples is used herein to compare Dexamethasone treatment with individual compounds and combinations as described above. Experiments are conducted as previously described in parallel to 3 groups of mice administered 1.5, 3 and 6 mg/kg/day of Dexamethasone. Survival is defined as the primary outcome.

In summary, we demonstrate that selective CB2 agonists improve the survival of septic mice, via their immunomodulatory actions that specifically target the innate immune system. Combining DHEA or Pregnenolone with OGP, Olorinab or BCP show a synergistic effect on the survival of LPS-injected mice.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-39. (canceled)

40. A pharmaceutical composition comprising:
at least one selective CB2 receptor agonist selected from the group consisting of: osteogenic growth peptide (OGP) (SEQ ID NO: 1), 5aa-OGP (SEQ ID NO: 2), olorinab, β-caryophyllene (BCP) and lenabasum; and
at least one steroid selected from the group consisting of: dehydroepiandrosterone (DHEA), dehydroepiandrosterone-sulfate (DHEAs), and pregnenolone.

41. A method of treating a pathogen infection and/or inflammation and/or associated complications, comprising administering to a subject in need of such treatment a therapeutically effective amount of:
(1) at least one selective CB2 receptor agonist selected from the group consisting of olorinab, β-caryophyllene (BCP), lenabasum, osteogenic growth peptide (OGP) (SEQ ID NO: 1), and a peptide comprising an amino acid sequence YGFGG (5aa-OGP; SEQ ID NO: 2), or an analog or derivative thereof; and
(2) at least one steroid selected from the group consisting of: dehydroepiandrosterone (DHEA), dehydroepiandrosterone-sulfate (DHEAs), and pregnenolone.

42. The method of claim 41, wherein the pathogen is a virus.

43. The method of claim 42, wherein the virus is a coronavirus.

44. The method of claim 41, wherein the subject suffers from sepsis and/or septic shock.

45. The method of claim 41, wherein the subject suffers from cytokine storm.

46. The method of claim 41, wherein the administering of the CB2 receptor agonist and the steroid is carried out substantially simultaneously, concurrently, alternately, sequentially or successively.

47. The method of claim 41, wherein the CB2 receptor agonist and the steroid are co-formulated.

48. The method of claim 48, wherein the CB2 receptor agonist and the steroid are co-formulated in a pharmaceutical composition further comprising at least one carrier, diluent or excipient.

49. The method of claim 41, wherein the peptide is selected from the group consisting of OGP (SEQ ID NO: 1) and 5aa-OGP (SEQ ID NO: 2).

50. The pharmaceutical composition of claim 40, comprising: 5aa-OGP (SEQ ID NO: 2) and at least one steroid selected from the group consisting of dehydroepiandrosterone (DHEA), dehydroepiandrosterone-sulfate (DHEAs) and pregnenolone.

51. The pharmaceutical composition of claim 40, comprising: OGP (SEQ ID NO: 1) and at least one steroid selected from the group consisting of dehydroepiandrosterone (DHEA), dehydroepiandrosterone-sulfate (DHEAs) and pregnenolone.

52. The pharmaceutical composition of claim 40, comprising: olorinab and at least one steroid selected from the group consisting of dehydroepiandrosterone (DHEA), dehydroepiandrosterone-sulfate (DHEAs) and pregnenolone.

53. The pharmaceutical composition of claim 52, comprising olorinab and DHEA.

54. The pharmaceutical composition of claim 40, comprising: ß-caryophyllene (BCP) and at least one steroid selected from the group consisting of dehydroepiandrosterone (DHEA), dehydroepiandrosterone-sulfate (DHEAs) and pregnenolone.

55. The pharmaceutical composition of claim 40, comprising: lenabasum and at least one steroid selected from the group consisting of dehydroepiandrosterone (DHEA), dehydroepiandrosterone-sulfate (DHEAs) and pregnenolone.

56. A method of treating inflammation and/or associated complications thereof comprising co-administering to a subject in need thereof: at least one selective CB2 receptor agonist selected from the group consisting of: OGP (SEQ ID NO: 1), 5aa-OGP (SEQ ID NO: 2), olorinab, β-caryophyllene (BCP) and lenabasum; and
at least one steroid selected from the group consisting of: dehydroepiandrosterone (DHEA), dehydroepiandrosterone-sulfate (DHEAs) and pregnenolone.

57. The method of claim 56, wherein the subject is infected with a pathogen.

58. The method of claim 56, wherein the at least one selective CB2 receptor agonist and the at least one steroid are in a single pharmaceutical composition.

59. The method of claim 56, wherein the subject suffers from post-infection symptoms or post-infection autoimmune disease.

60. A method of treating a subject infected with a pathogen, the method comprising the step of administering to the subject a pharmaceutical composition comprising a selective CB2 receptor agonist selected from the group consisting of: OGP (SEQ ID NO: 1), 5aa-OGP (SEQ ID NO: 2), olorinab, ß-caryophyllene (BCP) and lenabasum.
